(19) United States
(12) Patent Application Publication
(10) Pub. No.: US 2004/0122271 A1
(43) Pub. Date: Jun. 24, 2004

(54) Process for making a linear alpha-olefin oligomer using a heat exchanger

(76) Inventors: Arie Van Zon, Amsterdam (NL); Robert Moene, Amsterdam (NL); Phillip Edward Unger, Houston, TX (US); Peter Arnoldy, Amsterdam (NL); Eric Johannes Maria De Boer, Amsterdam (NL)

Correspondence Address: Donald F. Haas, Shell Oil Company, Legal - Intellectual Property, P.O. Box 2463, Houston, TX, 77252-2463, US

(21) Appl. No.: 10/668,934

(22) Filed: Sep. 23, 2003

Related U.S. Application Data
(60) Provisional application No. 60/413,276, filed on Sep. 25, 2002.

(51) Int. Cl. C07C 2/02; F28D 1/00

(52) U.S. Cl. 585/530; 422/198; 422/189

(57) ABSTRACT

The invention pertains to a process for making a linear alpha-olefin oligomer in a reactor comprising a liquid and a gas phase, comprising the steps of catalytically oligomerizing ethylene in the presence of a nickel, palladium, cobalt, titanium, zirconium, hafnium, vanadium, chromium, molybdenum or tungsten complex, to an alpha-olefin oligomer which preferably has an average molecular weight between 50 and 350 under release of heat, and removing the heat with a heat exchanger, which is not in direct contact with the liquid phase, using at least part of the gas phase as a coolant medium. The invention further relate to an apparatus to perform said process.

### FIELD OF THE INVENTION

The invention pertains to a process for making a linear alpha-olefin oligomer in a reactor comprising a liquid and a gas phase, comprising the steps of catalytically oligomerizing ethylene in the presence of a nickel, palladium, cobalt, titanium, zirconium, hafnium, vanadium, chromium, molybdenum, or tungsten complex, to the alpha-olefin oligomer with an average molecular weight of about 50 to about 350 under release of heat, and removing the heat with a heat exchanger.

### BACKGROUND OF THE INVENTION

Various catalysts and processes are known for the production of higher linear alpha olefins (for example W. Kaminsky and M. Arndt-Rosenau, Chemical Background in Applied Homogeneous Catalysis with organometallic Compounds, Ed. B. Cornils, W. A. Herrmann, 2^{nd} Edition, Vol. 1, Ch. 2.3.1.1, page 213-230, Wiley-VCH 2002 and D. Vogt, Oligomerisation of ethylene to higher alpha-olefins in Applied Homogeneous Catalysis with organometallic Compounds, Ed. B. Cornils, W. A. Herrmann, 2^{nd} Edition, Vol. 1, Ch. 2.3.1.1, page 240-253, Wiley-VCH 2002). The commercial processes afford either a Poisson or Schulz-Flory oligomer product distribution. In such a process, a wide range of oligomers is typically made.

For instance, British patent application GB 135,873 describes the preparation of C₄-C₂₀ linear alpha-olefins by ethylene oligomerization in the presence of a catalyst composition comprising a divalent nickel salt, a boron hydride, and a tertiary organophosphorus compound. PCT patent application WO 94/25416 discloses a catalyst system for the preparation of C₄-C₂₀ linear alpha-olefins comprising the reaction product of a bis-tetramethylcyclopentadienyl metallocene and a bulky, labile, and non-coordinating anion. PCT patent applications WO 96/27439 and WO 99/52631 describe a class of oligomerization catalysts comprising a bridged bis-amido Group 4 (IUPAC 1988 notation) metal compound, such as {1,2-bis(t-butylamide)tetramethyldisilane}zirconium dibenzyl or dimethyl, in association with suitable activating agents, capable of providing a bulky, labile and non-coordinating anion, such as B(C₆F₅)₃ or [Me₂PhNH]+[B(C₆F₅)₄]⁻.

Another process is the trimerization of ethylene to 1-hexene. Chromium-based catalysts are known to result in the principal formation of 1-hexene with more or less polyethylene, the proportion of butanes and octenes in the products being very low (R. M. Manyik, W. E. Walker, T. P. Wilson, J. Catal., 1977, 47, 197 and J. R. Briggs, Chem. Commun. 1989 and cited references). Catalysts for more or less selective ethylene trimerization have been claimed, for example in U.S. Pat. Nos. 5,198,563; 5,288,823; and 5,382,738; and in European patent publication Nos. 608447, 611743, and 0 614 865. Such catalysts are prepared from a chromium salt and a metallic amide, particularly a pyrrole. Other catalysts use an aluminoxane and a chromium complex with a chelating phosphine (U.S. Pat. No. 5,550,305 and WO 02/04119). These catalysts, which are incorporated by reference, are inter alia based on nickel, palladium, cobalt, titanium, zirconium, hafnium, vanadium, chromium, molybdenum, or tungsten complexes.

Alpha-olefin oligomers are compounds or a mixture of compounds with the general formula H₂C═CH—(CH₂CH₂)ₙH wherein n is an integer of 1 or greater. In such oligomers the alpha-olefin oligomer is usually a mixture of alpha-olefin oligomers with a mean number n from 1 to 20, preferably from 2 to 10. Alpha-olefin oligomers prepared according to the process of the present invention preferably have an average molecular weight between 50 and 350, more preferably between 60 and 280, even more preferably between 80 and 210.

The reaction of ethylene in the presence of one of the above complexes is usually run in a well-mixed reactor in the liquid phase, typically using an aprotic organic solvent. This reaction generates a large amount of heat, which should be removed. As described in WO 02/06192 it is preferred to install a plurality of small reactors in combination with several heat exchangers to help provide sufficient cooling capacity for the reactor system. The process temperature, which usually is between about 35° C. and about 90° C., more preferably between about 35° C. and about 75° C., affects the cost of manufacture of the alpha-olefins in several ways. The higher the temperature the smaller the heat exchangers which have to be applied to the reactor(s), which generally lowers cost. The decay of the active oligomerization catalyst increases with increasing temperature. It is found that maximum volumetric production of alpha-olefins coupled with good absolute productivity of the catalyst usually occurs in the range of about 45° C. to about 75° C., so this temperature range is preferred. Finally, the temperature also affects the bubble point pressure, the amount of ethylene in the liquid phase, and the catalyst selectivity. The higher the temperature the higher the pressure needed to maintain catalyst selectivity, which increases capital cost of the manufacturing plant because of, for example, the need for thicker vessels, and larger compressors to attain the higher ethylene pressure. Higher pressure also increases energy costs.

The amount of ethylene (ethene) oligomerization catalyst used in the reaction will preferably be the maximum permitted by the cooling capacity of the reactor(s) and the ethylene mass transfer from the gas to the liquid phase. Catalyst may be added to the first reactor only or to one or more subsequent reactors in series. Differing amounts of catalyst may be added to each reactor. The oligomerization is quite exothermic, about 100 kJ/mole of ethylene oligomerized, and as such cooling will usually be applied to the reactor(s) to maintain the desired process temperature while maintaining high volumetric productivity of the reactor(s).

In the prior art cooling is accomplished by running cooling tubes through the liquid in the interior of one or more of the reactors to cool the contents. Another method of cooling is to have one or more heat exchangers external to the reactors and connected to the reactors by a liquid loop to cool the reactor contents. These external heat exchangers may be typical shell and tube exchangers. The reactors may also be jacketed with a cooling jacket. Some or all of the feeds to some or all of the reactors may be cooled to allow the sensible heat of the ingredients to cool the reactors. All these liquid cooling methods, however, suffer from the disadvantage of wax and polyethylene fouling of the coolers, which necessitates regular shut down of the reactor to allow cleaning of the coolers. Furthermore, wax and polyethylene fouling may increase the paraffinicity of the solvent.

### SUMMARY OF THE INVENTION

It would therefore be advantageous to devise a process without the above disadvantages. It has now been found that linear alpha-olefin oligomers can be made in a reactor comprising a liquid and a gas phase, comprising the steps of catalytically oligomerizing ethylene in the presence of a nickel, palladium, cobalt, titanium, zirconium, hafnium, vanadium, chromium, molybdenum, or tungsten complex (preferably of a 2,6-bis(arylimino)pyridine derivative), to an alpha-olefin oligomer which preferably has an average molecular weight between about 50 and about 350 under release of heat, and removing the heat with a heat exchanger, which is not in direct contact with the liquid phase, using at least part of the gas phase as a coolant medium.

This method provides a cooling system having its cooling elements outside the liquid reaction medium. Since wax and polyethylene have high boiling points, deposit of wax and polyethylene can no longer occur, and fouling of the heat exchanger is effectively prevented.

### BRIEF DESCRIPTION OF THE DRAWINGS

The invention is illustrated by the following Figures, which are not meant to limit the invention in any way, showing a scheme of an apparatus that can be used for performing the process of the invention.

FIG. 1 is a scheme of an apparatus for performing the method according to the invention with the heat exchanger positioned outside the reactor.

FIG. 2 is a scheme of an apparatus for performing the method according to the invention with the heat exchanger positioned inside the reactor.

### DETAILED DESCRIPTION OF THE INVENTION

The heat exchanger according to this invention is of a conventional type, such as a shell- and tube-type, and the like. The heat exchanger is internally cooled with conventional cooling fluids, like water, ammonia, Freon® coolant, and the like. The reaction heat causes the solvents, reactants, and/or reaction products, which are present in the reaction medium, to evaporate and subsequently to be cooled by the heat exchanger, after which it works as a coolant medium for the reactor. The heat exchanger can be placed inside or outside the reactor. When the heat exchanger is placed inside the reactor it is preferred that some condensation occurs on the heat exchanger surface. When the heat exchanger is placed outside the reactor, it is preferred to apply a forced circulation of the reactor coolant medium from the gas phase of the reactor through heat exchanger(s) compressor(s)/pump(s) and optionally a gas-liquid separator back to the liquid phase of the reactor. This will additionally improve the mixing in the reactor. After cooling this reactor coolant medium in this loop, some condensation can occur. This allows application of a separate gas and liquid return to the reactor using a gas-liquid separator. Furthermore, it is possible to deliberately remove (part of) this liquid phase from this gas-liquid separator and route this directly to the product work-up section. Finally, if full condensation occurs, return of this liquid to the reactor can be achieved by a pump instead of a compressor, which lowers costs. This reactor coolant medium is selected from an alkane, inert heteroatom-containing group substituted alkane, alkene, and aromatic compound, and mixtures thereof. The terms alkane and alkene mean an unbranched or branched C1-C8 alkane and C2-C8 alkene, respectively. The alkane may be substituted with an inert heteroatom-containing group, wherein the term “inert” means that the heteroatom containing group, such as an O- or N-containing group does not react with the other components under the conditions used. The term aromatic compound means a homo- or heteroaromatic group with at least a 5-membered aromatic ring. Phenyl aromatic groups are preferred. The aromatic groups may be substituted with the common aromatic substituents such as alkyl, alkoxy, halide, and the like.

Preferred reactor coolants are selected from propane, n-pentane, isopentane, ethylene, 1-butene, o-, m-, and p-xylene, and toluene, and mixtures thereof.

An additional advantage of the present process is the possibility to apply only one reactor, because the efficiency and the lack of fouling no longer necessitates the use of a plurality of small reactors. This adds considerably to the lowering of costs of the oligomerization process.

The nickel, palladium, cobalt, titanium, zirconium, hafnium, vanadium, chromium, molybdenum, and tungsten complexes that can be used in the above process are known in the art, and are described in the previously mentioned patents and patent applications. Any of these complexes can be used. Preferred for use in the process herein are nickel, titanium, zirconium or chromium complexes. Most preferred are nickel catalyst compositions comprising a divalent nickel salt, a boron hydride, and a tertiary organophosphorus compound, a titanium or zirconium catalyst comprising the reaction product of a bis-tetramethylcyclopentadienyl metallocene and a bulky, labile, and non-coordinating anion, a titanium or zirconium catalyst comprising a bridged bis-amido Group 4 (IUPAC 1988 notation) metal compound, such as {1,2-bis(t-butylamide)tetramethyl-disilane}zirconium dibenzyl or dimethyl, in association with suitable activating agents, capable of providing a bulky, labile and non-coordinating anion, such as B(C₆F₅)₃ or [Me₂PhNH]+[B(C₆F₅)₄]⁻, and chromium complexes comprising the reaction product of a chromium salt and a metallic amide, particularly a pyrrole or comprising a chromium complex with a phosphine and an aluminoxane.

An important item in the capital cost of the manufacturing plant and in the cost of operating it is the amount of reactor coolant medium that must be recycled in the process. Recycling of a gaseous reactor coolant medium often involves recompression to feed one or more of the reactors. Compressors and associated equipment add greatly to capital and operational costs. In the present method the coolant medium is preferably selected to completely dissolve ethylene. In this case the coolant medium only requires a single reactor and a condenser, whereas a simple recycle pump is sufficient. Thus expensive recycling, such as the use of an expensive recycle blower, is no longer required, which adds further to the advantages of the present method.

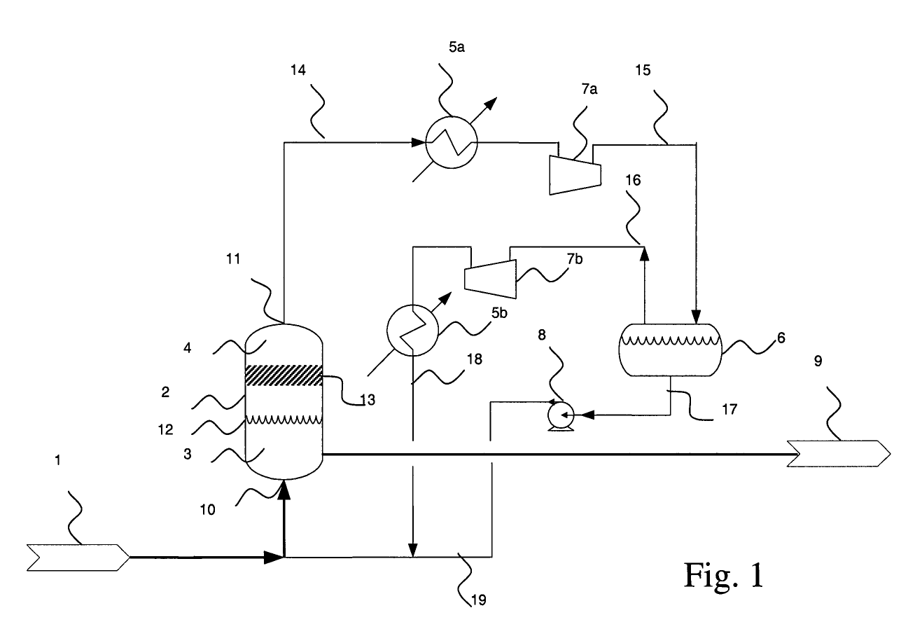

FIG. 1 shows a reactor 2 with a liquid phase 3 and a gas phase 4 being in equilibrium through gas/liquid interface 12. The liquid phase comprises ethylene, the nickel, palladium, cobalt, titanium, zirconium, hafnium, vanadium, chromium, molybdenum, or tungsten complex of a 2,6-bis(arylimino)pyridine derivative, alpha-olefin oligomer, and optionally solvents and auxiliaries such as a co-catalyst. The optional solvents are selected as to dissolve ethylene. The reactor 2 contains an inlet 10 through which the reactor feed 1 (usually ethylene) is introduced into the reactor 2, a gas outlet 11, and a reactor bottom outlet 9. In the embodiment of FIG. 1, outlet 11 is connected through a conduit 14 to heat exchanger 5a, which is connected through conduit 15 to gas-liquid separator 6. If necessary, conduit 15 may contain a compressor 7a. Gas-liquid separator 6 has an outlet for transporting the liquid, optionally through a pump 8, to obtain a pressurized liquid stream 17 that is recycled via conduit 19 to reactor 2. The gas leaves the gas-liquid separator 6 through conduit 16, which may optionally comprise compressor 7b and/or heat exchanger 5b, to obtain a cooled gas stream 18 that is recycled to reactor 2. If no condensation occurs in conduit 15, gas-liquid separator 6, and pump 8 are redundant and may be deleted. In that case conduit 15 can directly be connected to compressor 7b and/or heat exchanger 5b, if present, or to conduit 19. Reactor 2 may contain an optional entrainment separator 13.

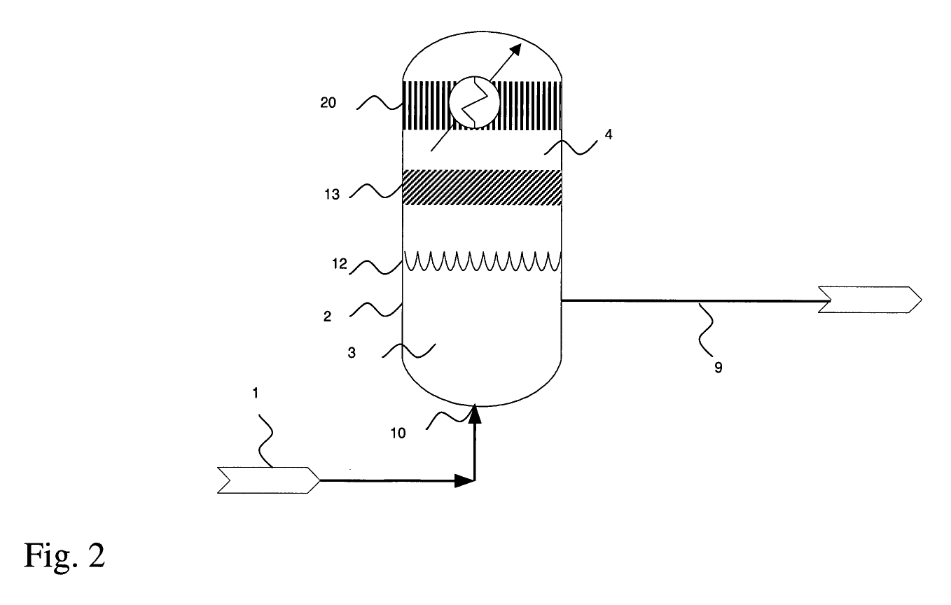

FIG. 2 shows another embodiment of the invention. In this embodiment the reactor feed 1 is introduced into the reactor 2 through inlet 10. The liquid phase 3 in the reactor is in equilibrium with the gas phase 4 through gas/liquid interface 12. In the section of the reactor containing the gas phase 4, a heat exchanger 20 is placed, which is not in contact with the liquid phase 3. The section of the gas phase 4 may optionally contain an entrainment separator 13. The heat exchanger 20 cools the gas, after which at least part of the gas condenses and the cooled condensate falls down from the surface of the heat exchanger 20 into the liquid phase 3, thereby cooling the liquid medium. The reaction product may then be discharged from the reactor through the reactor bottom outlet 9.

Hence, according to a further aspect of the present invention there is provided an apparatus for performing the process of making linear alpha-olefin oligomer described above, comprising a reactor, which can accommodate a liquid and a gas phase, an inlet through which the reactor feed can be introduced into the reactor, a reactor bottom outlet to remove the oligomer, and a heat exchanger, which is positioned in the gas phase to condense the gas and allow the condensate to fall therefrom to cool the liquid phase, and optionally, an entrainment separator, and/or a gas-liquid separator.

We claim:

1. A process for making a linear alpha-olefin oligomer in a reactor comprising a liquid and a gas phase, comprising the steps of catalytically oligomerizing ethylene in the presence of a complex selected from the group consisting of nickel, palladium, cobalt, titanium, zirconium, hafnium, vanadium, chromium, molybdenum, and tungsten complexes, to an alpha-olefin oligomer under release of heat, and removing the heat with a heat exchanger, which is not in direct contact with the liquid phase, using at least part of the gas phase as a coolant medium.

2. The process of claim 1 wherein the complex is selected from the group consisting of nickel, titanium, zirconium, and chromium complexes.

3. The process of claim 1 wherein the alpha olefin oligomer has an average molecular weight between about 50 and about 350.

4. The process of claim 3 wherein the average molecular weight is between about 60 and about 280.

5. The process of claim 4 wherein the average molecular weight is between about 80 and about 210.

6. The process of claim 1 wherein the coolant medium is selected from the group consisting of an alkane, an inert heteroatom-containing group substituted alkane, an alkene, and an aromatic compound, and mixtures thereof.

7. The process of claim 1 wherein the coolant medium is selected from the group consisting of propane, n-pentane, isopentane, ethylene, 1-butene, o-, m-, and p-xylene, and toluene, and mixtures thereof.

8. An apparatus for performing the process of making linear alpha-olefin oligomer of claim 1 comprising a reactor which can accommodate a liquid phase and a gas phase, an inlet through which the reactor feed is introduced into the reactor, a reactor bottom outlet through which the oligomer is removed, a heat exchanger which is positioned in the gas phase to condense the gas and allow the condensate to fall therefrom to cool the liquid phase thereby cooling the liquid, and optionally, a gas outlet and/or an entrainment separator.

9. The apparatus of claim 8 wherein a gas entrainment separator which is positioned in the gas phase.

10. An apparatus for performing the process of making linear alpha-olefin oligomer of claim 1 comprising 1) a reactor which can accommodate a liquid phase and a gas phase, a reactor feed inlet, a gas outlet, and a reactor bottom outlet for the reaction products, 2) a heat exchanger which is positioned outside of the reactor, receives gas from the reactor gas outlet, and cools the gas, wherein said gas flows from the heat exchanger through a first gas conduit where part of the gas condenses, 3) a gas-liquid separator which has a gas outlet and a liquid outlet, receives gas and liquid from the heat exchanger, and separates gas, which exits the separator through a second gas conduit and is recycled to the reactor, from liquid, which exits the separator through a liquid conduit and is recycled to the reactor.

11. The apparatus of claim 10 further comprising a compressor between the heat exchanger and the gas-liquid separator.

12. The apparatus of claim 11 further comprising a pump in the liquid conduit.

13. The apparatus of claim 11 further comprising a compressor and/or a heat exchanger in the second gas conduit.

14. The apparatus of claim 11 further comprising an entrainment separator in the reactor in the gas phase.

15. An apparatus for performing the process of making linear alpha-olefin oligomer of claim 1 comprising 1) a reactor which can accommodate a liquid phase and a gas phase, a reactor feed inlet, a gas outlet, and a reactor bottom outlet for the reaction products, and 2) a heat exchanger which is positioned outside of the reactor, receives gas from the reactor gas outlet, and cools the gas, wherein said gas flows from the heat exchanger through a gas conduit and is recycled to the reactor.

16. The apparatus of claim 15 further comprising a compressor and/or a heat exchanger in the gas conduit.
